(19) United States
(12) Patent Application Publication
(10) Pub. No.: US 2024/0090783 A1
(43) Pub. Date: Mar. 21, 2024

(54) Heart Rate Monitor Systems And Methods

(71) Applicant: Peloton Interactive, Inc., New York, NY (US)

(72) Inventors: Kah Jon GOO, New York, NY (US); Benjamin G. SCHULTZ, New York, NY (US); Sander CHANG, New York, NY (US); Otis CHU, New York, NY (US); Kevin KAN, New York, NY (US); Eric KISS, New York, NY (US); Megan MCLOUGHLIN, New York, NY (US); Jason OKAMOTO, New York, NY (US); Josh WU, New York, NY (US)

(21) Appl. No.: 18/264,366

(22) PCT Filed: Feb. 4, 2022
(86) PCT No.: PCT/US2022/015224

§ 371 Date: Aug. 4, 2023

Related U.S. Application Data
(60) Provisional application No. 63/146,436, filed on Feb. 5, 2021.

(51) Int. Cl. A61B 5/024; A61B 5/00

(52) U.S. Cl. CPC A61B 5/02427; A61B 5/6824; A61B 5/6831; A61B 5/742; A61B 5/7455; A61B 2560/0214; A61B 2560/0443; A61B 2560/0462; A61B 2562/0219

(57) ABSTRACT

A heart rate monitor may include a strap and a heart rate monitoring pod releasably coupled to the strap. A securement element may be disposed in the strap to selectively secure the pod to the strap. The pod may be selectively removable from the securement element and include one or more charger contacts. The pod may include one or more LEDs and/or vibrating elements to provide a light animation feedback and/or a haptic feedback to a user. A charger may be provided to charge the pod. The charger and pod may have complementary shapes to align the pod on the charger. For example, placement of the pod on the charger may align the charger contacts of the pod with one or more charging elements (e.g., pins) disposed in the charger.

### CROSS-REFERENCE TO RELATED APPLICATIONS

The present International PCT application claims priority to U.S. Provisional Patent Application No. 63/146,436, filed Feb. 5, 2021, the disclosure of which is hereby incorporated by reference in its entirety.

### TECHNICAL FIELD

One or more embodiments relate generally to health equipment and, more particularly, to systems and methods for a heart rate monitor.

### BACKGROUND

Heart rate monitors can be bulky and have one or more undesirable characteristics. For example, the heart rate monitoring device may be integrated with a strap, which may break, wear out, or become unsuited to a user's requirements, necessitating replacement of the entire heart rate monitoring device assembly. Some heart rate monitors use replaceable batteries that need to be replaced once depleted.

Therefore, there is a need in the art for systems and methods that address the deficiencies noted above, other deficiencies known in the industry, or at least offers an alternative to current techniques.

### SUMMARY

Systems and methods are provided for a heart rate monitor. According to one or more embodiments of the present disclosure, a heart rate monitor is provided. The heart rate monitor may include a strap including a securement element, and a pod releasably coupled to the securement element and configured to detect a heart rate of a user. The pod may be selectively removable from the securement element.

According to one or more embodiments of the present disclosure, a heart rate monitoring system is provided. The system may include a strap including a securement element, a pod coupled to the securement element and configured to detect a heart rate of a user, and a charger. The pod may be selectively removable from the securement element and include one or more charger contacts. The charger may include one or more pins configured to interface with the one or more charger contacts when the pod is coupled to the charger.

According to one or more embodiments of the present disclosure, a method is provided. The method may include coupling a pod of a heart rate monitor to a securement element disposed in a strap. The pod may be selectively removable from the securement element and configured to detect a heart rate of a user. The method may include measuring, using the pod, the heart rate of the user. The method may include engaging the pod with a charger to align one or more charger contacts of the pod with one or more pins of the charger.

Additional features are set forth in part in the description that follows and will become apparent to those skilled in the art upon examination of the specification and drawings or may be learned by the practice of the disclosed subject matter. A further understanding of the nature and advantages of the present disclosure may be realized by reference to the remaining portions of the specification and the drawings, which forms a part of this disclosure.

One of skill in the art will understand that each of the various aspects and features of the disclosure may advantageously be used separately in some instances, or in combination with other aspects and features of the disclosure in other instances. Accordingly, individual aspects can be claimed separately or in combination with other aspects and features. Thus, the present disclosure is merely exemplary in nature and is in no way intended to limit the claimed invention or its applications or uses. It is to be understood that structural and/or logical changes may be made without departing from the spirit and scope of the present disclosure.

The present disclosure is set forth in various levels of detail and no limitation as to the scope of the claimed subject matter is intended by either the inclusion or non-inclusion of elements, components, or the like in this summary. In certain instances, details that are not necessary for an understanding of the disclosure or that render other details difficult to perceive may have been omitted. Moreover, for the purposes of clarity, detailed descriptions of certain features will not be discussed when they would be apparent to those with skill in the art so as not to obscure the description of the present disclosure. The claimed subject matter is not necessarily limited to the arrangements illustrated herein, with the scope of the present disclosure is defined only by the appended claims.

### BRIEF DESCRIPTION OF THE DRAWINGS

The description will be more fully understood with reference to the following figures in which components may not be drawn to scale, which are presented as various embodiments of the seat assembly described herein and should not be construed as a complete depiction of the scope of the seat assembly.

FIG. 1 illustrates a heart rate monitoring system, in accordance with an embodiment of the disclosure.

FIG. 2 illustrates a heart rate monitor with a pod secured to a strap, in accordance with an embodiment of the disclosure.

FIG. 3 illustrates the heart rate monitor with the pod removed from the strap, in accordance with an embodiment of the disclosure.

FIG. 4 illustrates a right-side view of the pod, in accordance with an embodiment of the disclosure.

FIG. 5 illustrates a top view of the pod, in accordance with an embodiment of the disclosure.

FIG. 6 illustrates a left-side view of the pod, in accordance with an embodiment of the disclosure.

FIG. 7 illustrates a bottom view of the pod, in accordance with an embodiment of the disclosure.

FIG. 8 illustrates an exploded view of the pod, in accordance with an embodiment of the disclosure.

FIG. 9 illustrates an exploded view of a bottom housing of the pod, in accordance with an embodiment of the disclosure.

FIG. 10 illustrates an exploded view of the strap, in accordance with an embodiment of the disclosure.

FIG. 11 illustrates example exterior configurations of the strap, in accordance with an embodiment of the disclosure.

FIG. 12 illustrates example interior configurations of the strap, in accordance with an embodiment of the disclosure.

FIG. 13 illustrates a top view of a charger of the heart rate monitoring system, in accordance with an embodiment of the disclosure.

FIG. 14 illustrates a bottom view of the charger, in accordance with an embodiment of the disclosure.

FIG. 15 illustrates a side view of the charger, in accordance with an embodiment of the disclosure.

FIG. 16 illustrates an exploded view of the charger, in accordance with an embodiment of the disclosure.

FIG. 17 illustrates a flow diagram of a process of operating a heart rate monitoring system, in accordance with an embodiment of the disclosure.

Embodiments of the disclosure and their advantages are best understood by referring to the detailed description that follows. It should be appreciated that like reference numerals may be used to identify like elements illustrated in one or more of the figures.

### DETAILED DESCRIPTION

According to the present disclosure, heart rate monitoring systems and methods are provided. A heart rate monitor may include a strap and a heart rate monitoring pod releasably coupled to the strap. A securement element (e.g., a thermoplastic polyurethane (TPU) ring) may be disposed in the strap to selectively secure the pod to the strap. The pod may be selectively removable from the securement element and include one or more charger contacts. The pod may include one or more LEDs and/or vibrating elements to provide a light animation feedback and/or a haptic feedback to a user.

A charger may be provided to charge the pod. The charger and pod may have complementary shapes to align the pod on the charger. For example, placement of the pod on the charger may align the charger contacts of the pod with one or more charging elements (e.g., pins) disposed in the charger. The pod may be placed on the charger alone, or the charger may charge the pod while the pod is secured to the strap.

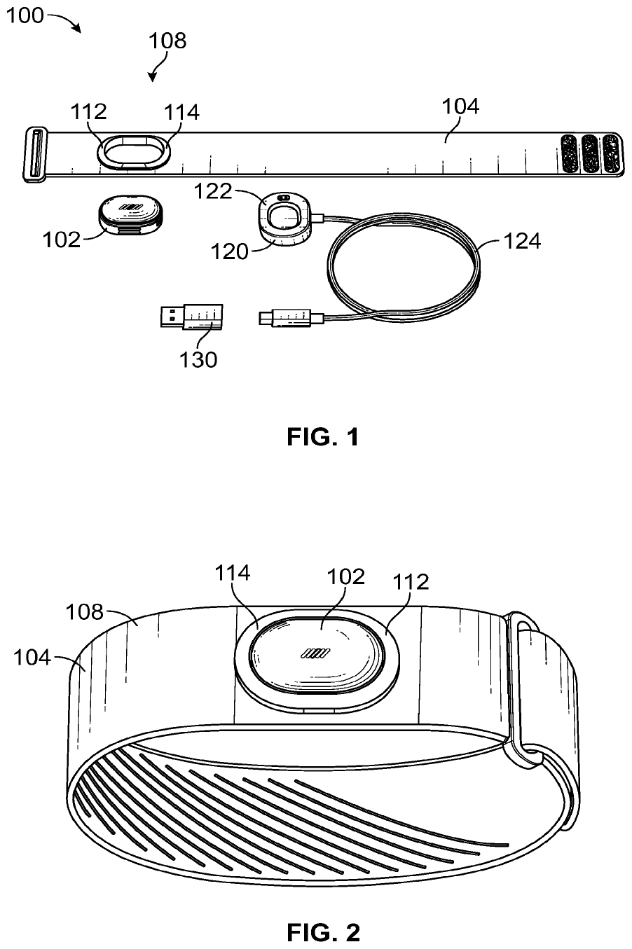

FIG. 1 illustrates a heart rate monitoring system, in accordance with an embodiment of the disclosure. Referring to FIG. 1, a heart rate monitoring system 100 includes a pod 102 or other device configured to detect a heart rate of a user. The pod 102 may allow the user to measure/display heart rate in real time and/or record the heart rate for later study. For example, the pod 102 may be configured to gather heart rate data while a user performs various types of physical exercise. The heart rate monitoring system 100 may include a strap 104. The strap 104 may be configured to secure the pod 102 on the user. For example, the strap 104 may wrap around a body part of the user to secure at least portions of the heart rate monitoring system 100 to the user. Depending on the application, the strap 104 may be placed as a wrist strap, such as the strap 104 wrapping around the user's wrist to secure the pod 102 near the user's distal radioulnar joint, although other configurations are contemplated. For instance, strap 104 may be placed anywhere on the arm of the user. Although described with reference to placement on the arm or wrist of the user, the strap 104 may be any strap adapted for placement on a user, such as a chest strap, a goggle strap, an ankle strap, a leg strap, a head strap, or other strap, for example. When secured together, the pod 102 and strap 104 may define a heart rate monitor 108.

As shown, the strap 104 may include a securement element 112 configured to capture or otherwise secure the pod 102 to the strap 104. For example, the securement element 112 may be a ring 114 (e.g., a TPU ring) disposed in the strap 104, such as nearer one of the ends of the strap 104. The ring 114 (or securement element 112) may be shaped complementary to the pod 102, such as having an ovular shape to match the ovular shape of the pod 102, as shown for example. As described more fully below, the pod 102 may be coupled to the securement element 112, such as releasably coupled to the securement element 112. For example, the pod 102 may fit within the securement element 112 to secure the pod 102 to the strap 104.

With continued reference to FIG. 1, the heart rate monitoring system 100 may include a charger 120. The charger 120 may be configured to charge one or more rechargeable batteries of the pod 102. For example, the pod 102 may be placed on the charger 120, whereupon the charger 120 charges the pod 102, as explained below. Depending on the application, the pod 102 may be charged wirelessly by the charger 120, or the pod 102 may be charged through one or more physical connections of the pod 102 to the charger 120, as described below. Depending on the application, the pod 102 may be charged by the charger 120 while the pod 102 is secured to the strap 104, or the pod 102 may need to be removed from the strap 104 for engagement with the charger 120.

As shown, the charger 120 may include a charging base 122 and a cord 124. The cord 124 may be fixed to the charging base 122, or the cord 124 may be removable from the charging base 122. For example, the cord 124 may be a USB cord, a USB-C cord, or the like, with a USB or USB-C connector of the cord 124 connectable to the charger base. In other embodiments, the cord 124 may be hardwired to the charging base 122. In some embodiments, the heart rate monitoring system 100 may include one or more adapters 130 configured to connect the cord 124 to legacy USB ports. For example, the adapter 130 may be a USB-C female to USB male adapter, although other configurations are contemplated.

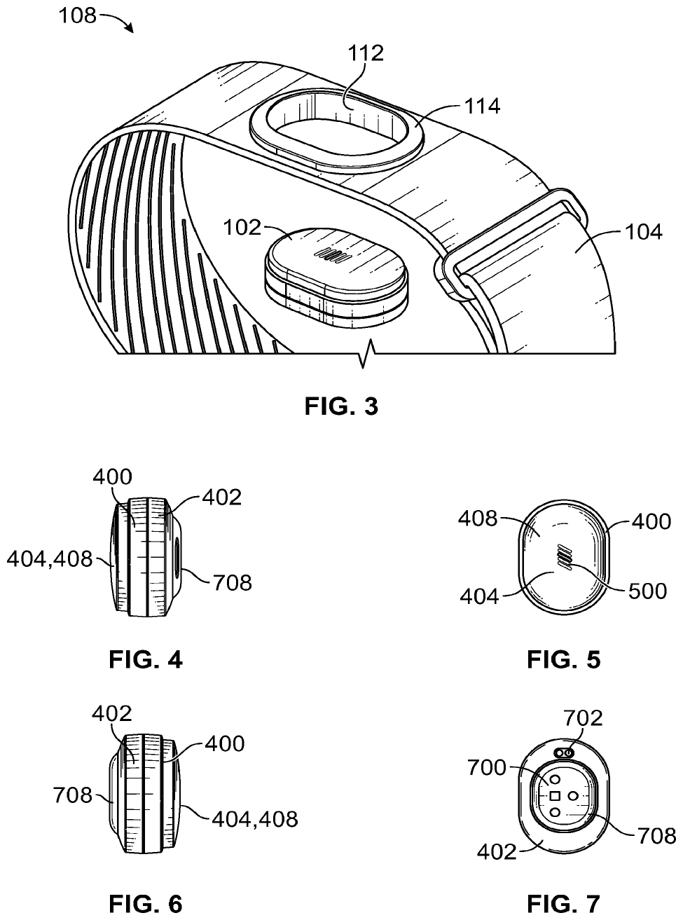

FIG. 2 illustrates the heart rate monitor 108 with the pod 102 secured to the strap 104, in accordance with an embodiment of the disclosure. FIG. 3 illustrates the heart rate monitor 108 with the pod 102 removed from the strap 104, in accordance with an embodiment of the disclosure. Referring to FIGS. 2-3, the pod 102 may be releasably coupled to the securement element 112 of the strap 104, with the pod 102 selectively removable from the securement element 112 as desired. For instance, as shown in FIG. 2, the pod 102 may be coupled to the securement element 112 to provide heart rate monitoring functionality when the strap 104 is secured to the wrist of a user. Referring to FIG. 3, the pod 102 may be removed from the securement element 112, such as to facilitate washing of the strap 104, charging of the pod 102, replacement of the pod 102 or strap 104, etc.

The pod 102 may be releasably coupled to the strap 104 (e.g., to the securement element 112) in many configurations. As one example, the pod 102 may be snap fitted to the ring 114 to releasably couple the pod 102 to the strap 104. For example, the interface between the pod 102 and the ring 114 may include one or more detents, corresponding retention features, or the like to the releasably couple the pod 102 to the ring 114, as described below. Referring to FIG. 3, the interface between the pod 102 and the securement element 112 may be configured to install and/or release the pod 102 in a particular direction. For example, the interface may be configured to install the pod 102 in a first direction, such as from a bottom of the strap 104 (i.e., by pressing the pod 102 from the bottom up). Similarly, the interface may be configured to remove the pod 102 in a second direction, such as from a top of the strap 104 (i.e., by pressing the pod 102 from the top down).

FIG. 4 illustrates a right-side view of the pod 102, in accordance with an embodiment of the disclosure. FIG. 5 illustrates a top view of the pod 102, in accordance with an embodiment of the disclosure. FIG. 6 illustrates a left-side view of the pod 102, in accordance with an embodiment of the disclosure. FIG. 7 illustrates a bottom view of the pod 102, in accordance with an embodiment of the disclosure. Referring to FIGS. 4-7, the pod 102 may include a top housing 400 and a bottom housing 402 secured together. The top housing 400, which may be referred to as a top cover, may define a top surface 404. In embodiments, the top surface 404 may define a button 408 (e.g., a single button 408). For example, deflection of the top surface 404 may activate the button 408, such as to control operation of the pod 102. The button 408 may be touch sensitive, an inductive button, or have any other suitable configuration. The button 408 may turn the pod 102 on and off and change the functionality/modes of the pod 102 as desired.

As best illustrated in FIG. 5, one or more light emitting diodes (LEDs 500) may be disposed in the top surface 404. The LEDs 500 may provide a light animation feedback. As shown, the pod 102 includes five LEDs 500, although the pod 102 may include greater or less than five LEDs 500. Each LED 500 or group of LEDs 500 may be associated with a particular characteristic, function, or use state of pod 102. For instance, each LED 500 may represent a different heart rate zone (e.g., rest, light, moderate, hard, maximum, etc.). Accordingly, illumination of a particular LED 500 may represent a detected heart rate Within an associated heart rate zone. In this way, users may, with a glance at the pod 102, quickly determine which heart rate zone they are in, in embodiments, illumination of one or more LEDs 500 may indicate a status of the pod 102, such as a charge status, an error status, a connection status, a communications status, a pairing status, or the like. Depending on the application, the light animation feedback (e.g., intensity, conditions precedent, etc.) may be preset or configured by the user as desired. Although LEDs 500 are described, the pod 102 may include any type of light emitting element.

As shown in FIG. 7, the bottom housing 402, which may be referred to as a bottom cover, may include a sensor lens 700 and one or more charger contacts 702 (e.g., a pair of charger contacts 702). The sensor lens 700 may be disposed in a sensor body 708 of the bottom housing 402 and is configured to detect a heart rate of a wearer of the heart rate monitor 108. The sensor body 708 may be shaped to interface with the charger 120 to align the pod 102 with the charger 120, as explained below. For example, the sensor body 708 may be shaped complementary to the charging base 122, so that placement of the sensor body 708 on the charger 120 aligns the pod 102 on the charging base 122. In embodiments, the sensor body 708 may be shaped to align the one or more charger contacts 702 of the pod 102 with the charger 120.

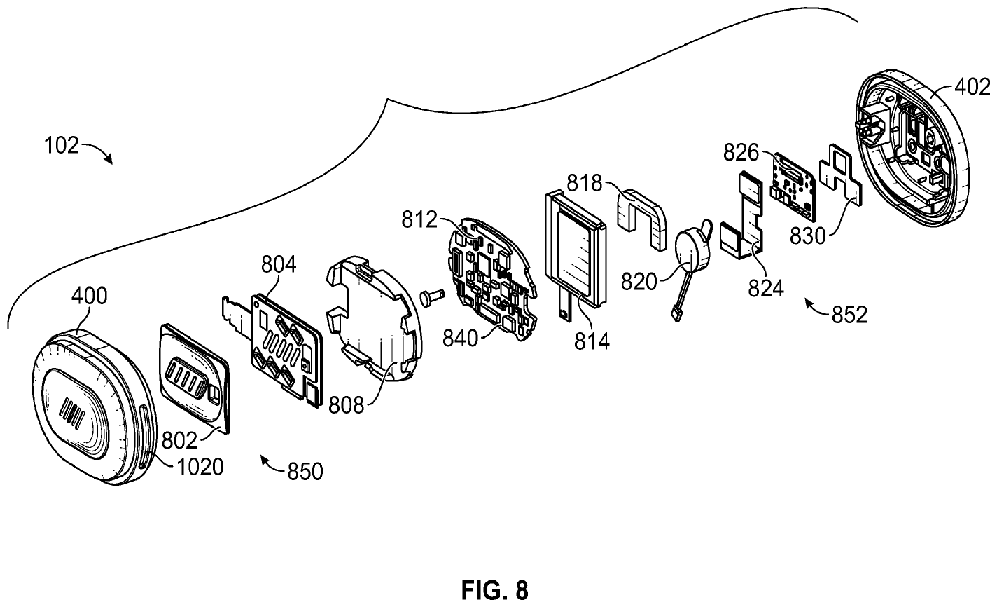

FIG. 8 illustrates an exploded view of the pod 102, in accordance with an embodiment of the disclosure. Referring to FIG. 8, the pod 102 may include top housing 400 and bottom housing 402 defining an internal cavity in which multiple components of the pod 102 are housed and contained. For example, the pod 102 may include an LED bracket assembly 802, an LED flexible printed circuit (LED FPC 804), an inner frame 808, a main printed circuit board assembly (main board 812), a battery 814, a sponge 818, a vibrator 820, a board-to-board flexible printed circuit (BTB FPC 824), a photoplethysmography sensor (PPG sensor 826), and a pressure sensitive adhesive (PSA 830), or any combination thereof, positioned within the internal cavity. The illustrated components are exemplary only, and the pod 102 may include other components. For instance, the pod 102 may include an ambient light sensor (e.g., to adjust light output of the LEDs 500) and/or a gyroscope, among other components.

The LED bracket assembly 802 may include the LEDs 500, and the LED FPC 804 may drive the LEDs 500 during operation. The main board 812 may include the necessary chipsets, memory, communications, or circuitry for operation of pod 102. For instance, the pod 102 may include one or more communication modules allowing wireless communication between the pod 102 and a network, a smart device (e.g., a user's smartphone, tablet, etc.), or a host machine (e.g., a stationary bicycle, a treadmill, etc.). For example, the pod 102 may be configured to pair with a user's smart device or a host machine via Bluetooth, Wi-Fi, or other wireless connection means. The pod 102 and smart device/host machine may communicate wirelessly with each other, such as the pod 102 providing heart rate data to the smart device/host machine for analysis, display, etc., or the smart device/host machine (e.g., an application running on the smart device/host machine) sending one or more commands to the pod 102 (e.g., to change an operation mode of pod 102, to run a haptic or LED animation feedback, etc.). In embodiments, main board 812 may include an inertial measurement unit (IMU 840) configured to detect a movement of the pod 102. For example, the IMU 840, which may have a 6-axis configuration, may be used to detect a rep completion, a step, or other movement of user.

The vibrator 820 may be configured to provide a haptic feedback. For example, the vibrator 820 may vibrate to indicate a status of pod 102, such as a charge status, an error status, a connection status, a communications status, a pairing status, or the like. As exemplary illustrations, the vibrator 820 may vibrate at the completion of a detected movement (e.g., after a number of reps, steps, etc.), upon reaching a particular heart rate zone, upon completing a desired milestone, at a particular charge status (e.g., 20% battery life remaining, 10% battery life remaining, etc.), or the like. In embodiments, the vibrator 820 may provide haptic feedback indicating the button 408 is pressed. Depending on the application, the haptic feedback (e.g., intensity, conditions precedent, etc.) may be preset or configured by the user as desired.

The PPG sensor 826 may be used to detect a heart rate using optical measurements. For example, the PPG sensor 826 may be used to detect blood volume changes in the microvascular bed of tissue (e.g., near a user's wrist). The PPG sensor 826 may use a pulse oximeter that illuminates the skin and measures changes in light absorption, such as monitoring the perfusion of blood to the dermis and subcutaneous tissue of the skin, although other configurations are contemplated. In embodiments, the PPG sensor 826 may illuminate the skin with an LED and then measure the amount of light either transmitted or reflected to a photodiode.

The various components of the pod 102 may be assembled together in many configurations. For example, the LED FPC 804 may be assembled to the LED bracket assembly 802 and positioned within the top housing 400, whereupon the inner frame 808 may be secured within the top housing 400 to secure the LED FPC 804 and LED bracket assembly 802 in place and define a top cover assembly 850. The PSA 830 may be secured in the bottom housing 402 to secure the PPG sensor 826 in place. The vibrator 820 may be placed in the bottom housing 402 adjacent to the PPG sensor 826 and the sponge 818 placed on top of the PPG sensor 826. The battery 814 and BTB FPC 824 may be positioned within the bottom housing 402, with the BTB FPC 824 connected to the PPG sensor 826 and the battery 814 positioned above the sponge 818. The main board 812 may be secured to the bottom housing 402 (e.g., via fasteners), with the battery 814 and BTB FPC 824 connected to the main board 812, to define a bottom cover assembly 852. The top cover assembly 850 may be assembled to the bottom cover assembly 852, such as the LED FPC 804 connected to the main board 812 and the top housing 400 coupled to the bottom housing 402 (e.g., via ultrasonic welding, adhesive, corresponding retention features, etc.) to define the completed pod 102. Once assembled, the pod 102 may have an IP7 ingress rating.

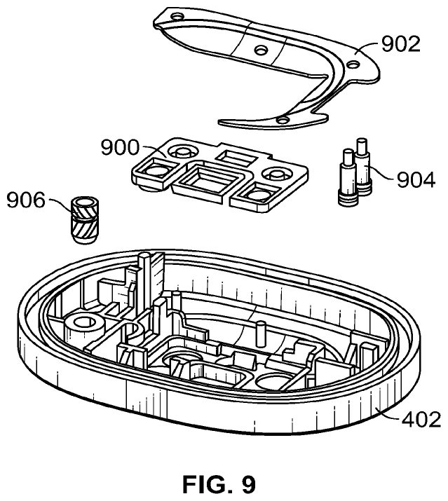

FIG. 9 illustrates an exploded view of the bottom housing 402, in accordance with an embodiment of the disclosure. Referring to FIG. 9, the pod 102 may include a photoplethysmography lens (PPG lens 900), a metal plate 902, a pair of pogo pins 904, and a nut 906 secured to the bottom housing 402. Each of the metal plate 902 and the nut 906 may be assembled to the bottom housing 402 via heat stacking, although other configurations are contemplated. The pogo pins 904 may define the charger contacts 702 and be overmolded into the bottom housing 402. During assembly of pod 102, the PPG sensor 826 may be coupled to the PPG lens 900, the pogo pins 904 may be coupled to the main board 812, and a fastener may secure the main board 812 to the nut 906.

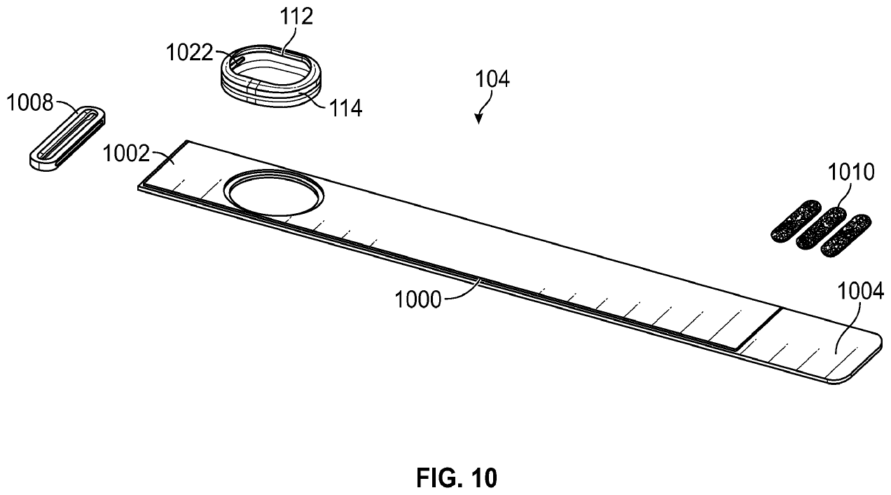

FIG. 10 illustrates an exploded view of the strap 104, in accordance with an embodiment of the disclosure. Referring to FIG. 10, the strap 104 may include a material strip 1000 having a first end 1002 and a second end 1004, the securement element 112 disposed in the material strip 1000, a clasp 1008 secured to the first end 1002, and one or more hook-and-loop fastener pads 1010 disposed on the second end 1004. The material strip 1000 may be formed from a flexible material, such as a textile fabric. The clasp 1008, which may be overmolded to the first end 1002 of the material strip 1000, may be configured to receive the second end 1004 of the strap 104 to loop the strap 104 around the wrist of the user. The hook-and-loop fastener pads 1010 may allow adjustment of the strap 104 to the user. For example, the hook-and-loop fastener pads 1010 may engage the material strip 1000 as desired to size the strap 104 to the user. The hook-and-loop fastener pads 1010 may be secured to the second end 1004 in many configurations, such as via stitching, ultrasonic welding, or the like.

Referring to FIGS. 8 and 10, the pod 102 and ring 114 may include a detent structure to releasably secure the pod 102 to the ring 114. For example, the top housing 400 may include a recess 1020 (see FIG. 8) shaped to interface with a detent 1022 disposed in the ring 114 (see FIG. 10), or vice versa. In this manner, the pod 102 may be snap-fitted to the ring 114 to releasably couple the pod 102 to the strap 104, although other configurations are contemplated.

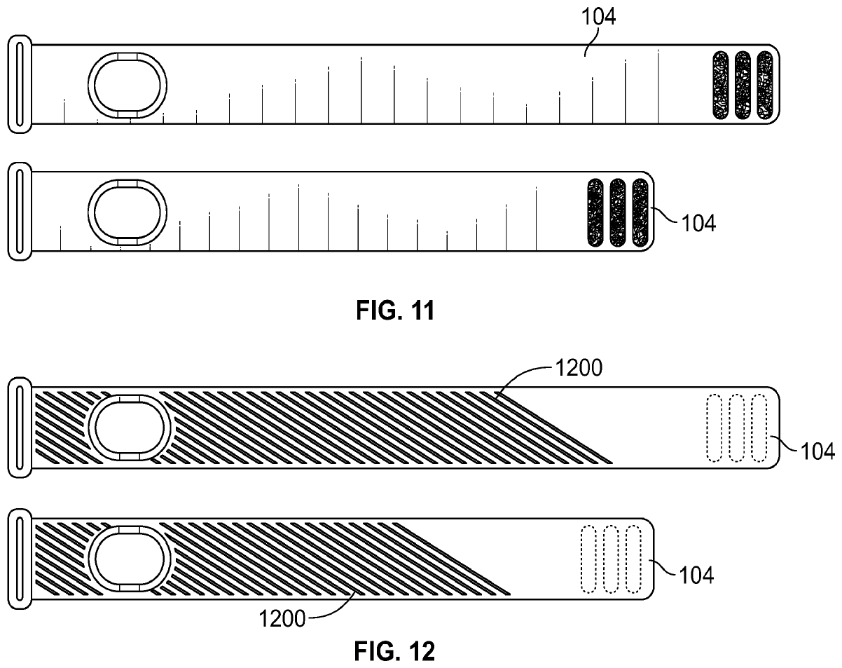

FIG. 11 illustrates example exterior configurations of the strap 104, in accordance with an embodiment of the disclosure. FIG. 12 illustrates example interior configurations of the strap 104, in accordance with an embodiment of the disclosure. Referring to FIGS. 11-12, the strap 104 may come in various sizes and configurations. For example, the strap 104 may have various lengths to suit the user, such as having a longer length (top straps shown in FIGS. 11-12) to suit users with larger wrists, or a shorter length (bottom straps shown in FIGS. 11-12) to suit users with smaller wrists. As such, the strap 104 may be replaced with a different strap of a different length, color, material, or another characteristic. In this regard, the pod 102 may be interchangeable with multiple straps.

Referring to FIG. 12, the strap 104 may include silicone rubber 1200 printed on the interior surface of the material strip 1000. The silicone rubber 1200 may provide a friction characteristic to grip the user's wrist. The silicone rubber 1200 may be printed in a desired pattern or design. For example, the silicone rubber 1200 may be printed on the interior surface, such as near the clasp 1008. The interior surface near the second end 1004 of the strap 104 may be free of silicone rubber 1200 to facilitate looping of the second end 1004 through the clasp 1008. The silicone rubber 1200 may have various colors and dimensions. For example, the silicone rubber 1200 may have a color different than strap 104 to distinguish the silicone rubber 1200 from the material strip 1000. In embodiments, the silicone rubber 1200 may project from the interior surface of the strap 104, such as about 0.3 mm, for instance. Although described with reference to silicone rubber printed on material strip 1000, elements 1200 may be formed from other materials and formed on material strip 1000 via other methods.

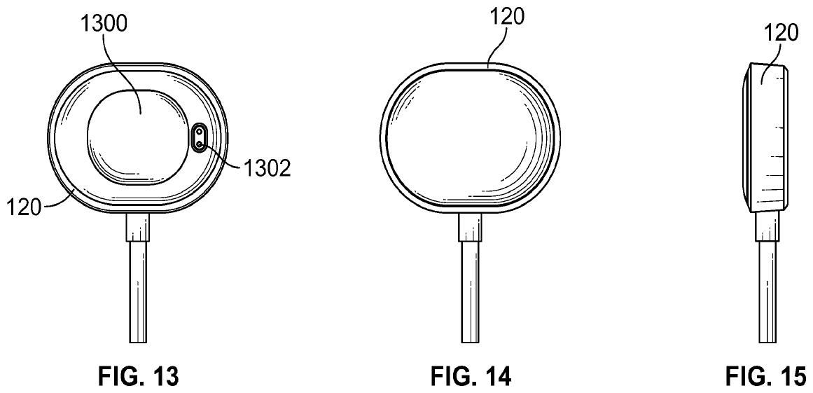

FIG. 13 illustrates a top view of the charger 120, in accordance with an embodiment of the disclosure. FIG. 14 illustrates a bottom view of the charger 120, in accordance with an embodiment of the disclosure. FIG. 15 illustrates a side view of the charger 120, in accordance with an embodiment of the disclosure. Referring to FIG. 13, the charger 120 may include a dished recess 1300 and one or more pins 1302 (e.g., a pair of pins 1302) positioned adjacent to the dished recess 1300. The dished recess 1300 may be shaped to interface with the sensor body 708 of the pod 102, such as to align the pod 102 with the charger 120. For example, the dished recess 1300 may be shaped complementary to the sensor body 708. The pins 1302 may be configured to interface with the one or more charger contacts 702 of the pod 102 when the pod 102 is coupled to the charger 120. For instance, placement of the pod 102 on the charger 120 may engage the charger contacts 702 with the pins 1302 to charge the pod 102. The complemental), shapes of the dished recess 1300 and the sensor body 708 may align the charger contacts 702 with the pins 1302 when the pod 102 is placed on the charger 120.

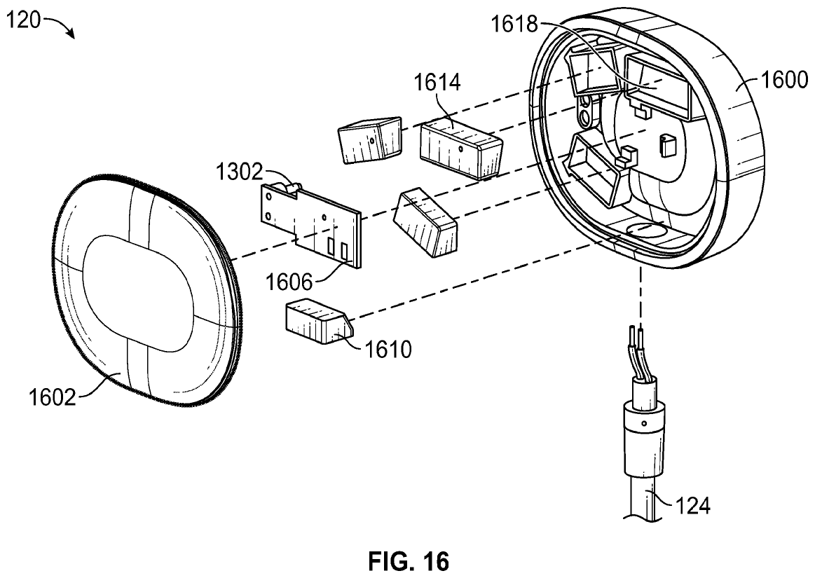

FIG. 16 illustrates an exploded view of the charger 120, in accordance with an embodiment of the disclosure. Referring to FIG. 16, the charging base 122 may include a top cover 1600 and a bottom cover 1602 secured to the top cover 1600. As shown, the charger 120 may include a charger board 1606 having the pins 1302. The cord 124 may be secured to the top cover 1600, such as via a cord connector 1610, and connected to the charger board 1606. The bottom cover 1602 may be bonded to the top cover 1600, such as via ultrasonic welding.

With continued reference to FIG. 16, one or more magnets 1614 may be secured to the top cover 1600, such as within corresponding recesses 1618 defined in the top cover 1600. Three magnets 1614 are shown, but the charger 120 may include greater or less than three magnets 1614 as desired. The magnets 1614 may be configured to magnetically couple the pod 102 to the charger 120. For example, the magnets 1614 may magnetically engage the metal plate 902 of the pod 102, pulling the pod 102 into the dished recess 1300 of the charging base 122 to secure the pod 102 to the charger 120. The magnetic engagement of the pod 102 with the charger 120 may maintain engagement of the charger contacts 702 of the pod 102 with the pins 1302 of the charger 120. For example, the magnets 1614 may be arranged asymmetrically within the top cover 1600 to magnetically engage the metal plate 902 in a single orientation (e.g., via an asymmetric metal plate 902) that aligns the charger contacts 702 with the pins 1302.

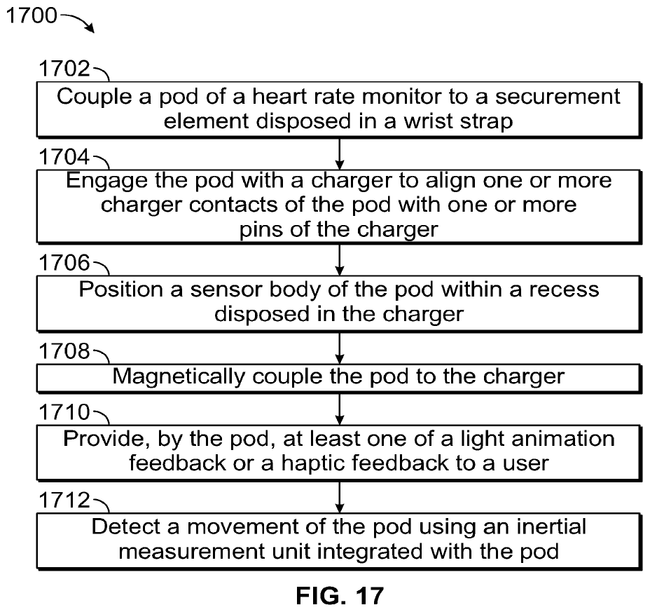

FIG. 17 illustrates a flow diagram of a process 1700 of operating a heart rate monitoring system, such as heart rate monitoring system 100, in accordance with an embodiment of the disclosure. Any step, sub-step, sub-process, or block of process 1700 may be performed in an order or arrangement different from the embodiments illustrated in FIG. 17. For example, one or more blocks may be omitted from or added to the process 1700. Although process 1700 is described with reference to the embodiments of FIGS. 1-16, process 1700 may be applied to other embodiments.

In block 1702, process 1700 includes coupling a pod of a heart rate monitor to a securement element disposed in a strap. The pod, securement element, and strap may be similar to pod 102, securement element 112, and strap 104 described above. For example, the pod may be selectively removable from the securement element and configured to detect a heart rate of a user. The securement element may be a ring (e.g., a TPU ring) disposed in the strap. The pod may be coupled to the securement element using a releasable coupling mechanism. The releasable coupling mechanism may secure the pod to the securement element from a first direction, such as by pressing the pod upwards from the bottom of the strap and into the securement element. In like manner, the releasable coupling mechanism may disengage the pod from the securement element from a second direction, such as by pressing the pod downwards from the top of the strap and out of the securement element. The releasable coupling mechanism may be a snap fitting or other releasable mechanism. For example, a recess may be disposed in the pod to receive a detent formed in the securement element.

In block 1704, process 1700 may include engaging the pod with a charger to align one or more charger contacts of the pod with one or more pins of the charger. For example, the pod may be removed from the strap for connection to the charger. In embodiments, the pod may be engaged with the charger while the pod is coupled to the strap. When the pod is placed on the charger, the pod may be oriented (e.g., automatically) such that the charger contact(s) of the pod align (e.g., naturally) with the pin(s) of the charger. In block 1706, the engaging may include positioning a sensor body of the pod within a recess disposed in the charger. The sensor body and recess may have complementary shapes to align the pod with the charger.

In block 1708, process 1700 may include magnetically coupling the pod to the charger. For example, one of the pod or the charger (e.g., the charger) may include one or more magnets and the other of the pod or the charger (e.g., the pod) may include a metal plate attracted to the magnet(s) via magnetism. The magnetic engagement of the pod to the charger may align and maintain engagement of the charger contacts of the pod with the pins of the charger. For example, the magnetic engagement may pull the pod into aligned engagement with the charger. As noted above, the magnets may be arranged asymmetrically within the charger to orient the pod for proper engagement of the charger contacts with the pins.

In block 1710, process 1700 may include providing, by the pod, at least one or a light animation feedback or a haptic feedback to a user. For instance, the pod may include one or more light emitting elements (e.g., five LEDs, etc.) configured to illuminate based on a characteristic, function, or use state of the pod. For example, the light emitting elements may indicate the detected heart rate within a heart rate zone, among other light animation feedback, as noted above. Additionally, or alternatively, the pod may include a vibrator configured to provide a “buzz” notification to a user as desired, such as upon completing a desired milestone or when a button of the pod is pressed, among others, as noted above.

In block 1712, process 1700 may include detecting a movement of the pod using an inertial measurement unit (IMU) integrated with the pod. For instance, the IMU may be used to detect a rep completion, a step, or other movement while the heart rate monitor is being worn by the user.

All relative and directional references (including up, down, upper, lower, top, bottom, side, front, rear, and so forth) are given by way of example to aid the reader's understanding of the examples described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that Iwo elements are directly connected and in fixed relation to each other, unless specifically set forth in the claims.

The present disclosure teaches by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall there between.

1. A heart rate monitor comprising:
a strap comprising a securement element; and
a pod releasably coupled to the securement element and configured to detect a heart rate of a user.

2. The heart rate monitor of claim 1, wherein:
the securement element is a ring disposed in the strap; and
the pod is snap fitted to the ring to releasably couple the pod to the strap.

3. The heart rate monitor of claim 1, further comprising:
an interface between the pod and the securement element, wherein the interface is configured to receive the pod from a bottom of the strap and release the pod from a top of the strap.

4. The heart rate monitor of claim 1, wherein the pod comprises:
a top housing comprising a button; and
a bottom housing comprising a sensor lens and one or more charger contacts.

5. The heart rate monitor of claim 4, wherein the sensor lens is disposed in a sensor body shaped to interface with a charger to align the one or more charger contacts of the pod with the charger.

6. The heart rate monitor of claim 4, wherein:
a top surface of the pod defines the button, such that deflection of the top surface activates the button; and
one or more light emitting elements are disposed in the top surface to provide light animation feedback to the user.

7. The heart rate monitor of claim 1, wherein the pod comprises at least one of a vibrator configured to provide haptic feedback to the user or an inertial measurement unit configured to detect a movement of the pod.

8. The heart rate monitor of claim 1, wherein the strap comprises:
a clasp secured to a first end of the strap and configured to receive an opposing second end of the strap to loop the strap around a wrist of the user; and
one or more hook-and-loop fastener pads disposed on the second end of the strap.

9. (canceled)

10. A system, comprising
a strap comprising a securement element;
a pod coupled to the securement element, wherein the pod comprises:
a sensor lens that detects a heart rate of a user and
one or more charger contacts; and
a charger comprising one or more pins configured to interface with the one or more charger contacts when the pod is coupled to the charger.

11. The system of claim 10, wherein the pod comprises a bottom housing comprising the sensor lens and the one or more charger contacts, the bottom housing shaped to interface with the charger to align the one or more charger contacts with the one or more pins of the charger.

12. The system of claim 11, wherein:
the charger comprises a dished recess; and
the sensor lens is disposed in a sensor body of the bottom housing, the sensor body shaped to interface with the dished recess to align the pod with the charger.

13. The system of claim 10, wherein the charger comprises one or more magnets configured to magnetically couple the pod to the charger, a magnetic engagement of the pod with the charger maintaining engagement of the one or more charger contacts of the pod with the one or more pins of the charger.

14. The system of claim 10, wherein the pod comprises:
a snap fitting to releasably couple the pod to the securement element;
a top surface defining a single button;
one or more light emitting diodes disposed in the top surface to provide light animation feedback to the user;
a vibrator configured to provide haptic feedback to the user; and
an inertial measurement unit configured to detect a movement of the pod.

15. The system of claim 10, wherein the strap comprises:
a clasp secured to a first end of the strap and configured to receive an opposing second end of the strap to loop the strap around a wrist of the user;
one or more hook-and-loop fastener pads disposed on the second end of the strap; and
silicone rubber printed on an interior surface of the strap.

16. A method comprising:
coupling a pod of a heart rate monitor to a securement element disposed in a strap worn by a user, the pod selectively removable from the securement element; and
measuring, using the pod, a heart rate of the user.

17. The method of claim 16, further comprising:
engaging the pod with a charger to align one or more charger contacts of the pod with one or more pins of the charger,
wherein the engaging comprises positioning a sensor body of the pod within a recess disposed in the charger, the sensor body and recess having complementary shapes to align the pod with the charger.

18. The method of claim 16, further comprising:
decoupling the pod from the securement element;
magnetically coupling the pod to a charger, wherein a magnetic engagement aligns and maintains engagement of one or more charger contacts of the pod with one or more pins of the charger;
recoupling the pod to the securement element; and remeasuring, using the pod, the heart rate of the user.

19. The method of claim 16, further comprising:
providing, by the pod, light animation feedback to the user that represents the measured heart rate of the user.

20. (canceled)

21. The method of claim 16, further comprising:
providing, by the pod, light animation feedback to the user that represents a current heart rate zone of the user associated with the measured heart rate of the user.

22. The method of claim 16, further comprising:
providing, by the pod, haptic feedback to the user that represents the measured heart rate of the user.
